(19) United States
(12) Patent Application Publication
(10) Pub. No.: US 2020/0046882 A1
(43) Pub. Date: Feb. 13, 2020

(54) Nano-Engineered Bioresorbable Polymer Composite for Bone-Soft Tissue Fixation Application

(71) Applicants: Indian Council of Medical Research, New Delhi (IN); Indian Institute of Technology, Bombay, Mumbai (IN)

(72) Inventors: Ajay Suryavanshi, Mumbai (IN); Kunal Khanna, Mumbai (IN); Jayesh Bellare, Mumbai (IN); Rohit Srivastava, Mumbai (IN)

(21) Appl. No.: 16/092,918

(22) PCT Filed: Apr. 13, 2017
(86) PCT No.: PCT/IN2017/000079

§ 371 Date: Oct. 11, 2018

(30) Foreign Application Priority Data
Apr. 13, 2016 (IN) 201611012973

(51) Int. Cl. A61L 27/44; A61L 27/48; A61L 27/36

(52) U.S. Cl. CPC A61L 27/446; A61L 2430/02; A61L 27/3604; A61L 27/48

(57) ABSTRACT

Provided herein is a novel bioresorbable polymer composite for bone soft tissue fixation including i) Silk fibroin in an amount of 5 to 30%, ii) a bioresorbable polymer matrix in an amount of 40 to 90%, and iii) magnesium oxide or other ceramic fillers in an amount of 5 to 30%.

### FIELD OF INVENTION

The invention relates to a bioresorbable polymer-composite based orthopedic fixation device or more particularly nano-composite biomaterial for bone-soft tissue fixation which is used to cater fixation of various bone and soft tissue injuries.

### BACKGROUND AND PRIOR ART OF THE INVENTION

A recent study showed that fractures account for an estimated 10.2 million visits a year to hospitals and physician offices in the US alone. Out of these, around 60% are fixed with osteosynthesis procedures utilizing various bone fixation devices (Centre for disease control and prevention). When we look at the soft-tissue fixation surgeries such as rotator cuff repairs, small joint fixations, meniscal repairs, cruciate ligament fixations, etc., total number of each in US alone is estimated to be around 2,00,000 to 3,00,000 per year. Therefore, there is an increasing demand for orthopedic devices, E.g. it is estimated that more than 100 million screws are used for bone or soft-tissue fixations per year.

Initially metallic devices (made of stainless steel, titanium alloy, cobalt-chrome alloy) were introduced for internal and external fixations of bone fractures. These are generally in the form of plates, screws, rods, pins, wires, intramedullary nails, etc. The major problems with such devices are revision surgery and stress shielding. Subsequently, polymeric devices (composed of PLLA, PLGA, etc.) came to market for obviating need of revision surgery by being resorbable. However, such devices also suffer from certain disadvantages viz. inadequate mechanical properties, poor bioactivity, longer degradation period and release of acidic degradation by products responsible for inflammatory reactions. To overcome said shortcomings, biocomposite devices were developed which comprises polymer and bioactive filler viz. HA, β-TCP, etc. which improves mechanical strength, neutralizes acidic byproducts and enhances its bioactivity and degradation rate. The widely used biocomposite compositions for making orthopedic devices are combinations of PLLA, PLGA, PLDLA with HA, β-TCP etc.

Some patents related to such orthopedic bioresorbable composites are mentioned in the art.

A list of such patents is given below in table 1.

Some of the non-patent literature reports list has also been cited below (Table 2).

TABLE 2

| Sr. | | Mixture | | | | |
|---|---|---|---|---|---|---|
| No. | Title | composition | Parameters | Properties | Application | Reference |
| 1 | Prediction of | PLA/HA | Phyalcochemical | Retains mechanical | Used for lag | David D. Hile |
| | resorption rates | 75% PLA and | evaluation of In- | integrity during | screws | et al 2003 |
| | for composite | 25% HA | vitro degradation, | polymer | | |
| | polyactide/ | | Mechanical | degradation, | | |
| | hydroxyapatite | | evaluation | HA is | | |
| | internal fixation | | | osteoconductive, | | |
| | devices initial | | | controls acidic | | |
| | degradation | | | byproducts without | | |
| | profiles | | | changing | | |
| | | | | mechanical | | |
| | | | | properties | | |
| 2 | Preparation and | PLLA/HA | Mechanical | Filler | Scaffold for | K. Kesenc et al |
| | properties of | 90%/10% | properties and | reinforcement, | regeneration of | 2015 |
| | poly(L- | 70%/30% | degradation | Improves retention | bone tissue, | |
| | lactide)/hydroxyapatite | 50%/50% | behavior | characteristics | construction of | |
| | composites | | | | load bearing | |
| | | | | | componants | |
| 3 | Mechanical | Poly lactic acid | Tensile strength | Mechanical | Designing of the | Mei-po Ho et |
| | Properties of an | and 5% wt silk | Flexural strength | improvement, | biodegradable | al 2010 |
| | Injected Silk | fibroin | | biocompatible | plates | |
| | Fiber Reinforced | | | | | |
| | PLA | | | | | |
| | Composite | | | | | |
| 4 | Characteriatics | 5% wt Silkworm | Tensile strength | Mechanical and | Used in bio- | Mei-po Ho et |
| | of a Silk Fiber | fibers and poly | | thermoplastic | engineering and | al 2010 |
| | Reinforced | lectic acid | | properties | tissue | |
| | Biodegradable | | | improved | engineering | |
| | Plastic | | | | applications. | |
| 5 | Biodegradability | Poly lactic acid | Biodegradation | Mechanical and | Bioengineering | H. Y. cheung et |
| | of a silkworm | and silk fiber | test and | biodegradable with | process of the | al 2012 |
| | silk fiber | Comparison | Analysis of silk | high crystalline | regeneration of | |
| | reinforced | between tussah | fibers | structure | neo-tissues | |
| | poly(lactic add) | and Bombyx mori | SEM to observe | | | |
| | biocomposite | silk fibers | the morphology of | | | |
| | | | two kinds of fiber | | | |
| 6 | Characterization | 5% Silk fibers and | Mechanical | Specific strength | Prosthetic | H. Y. cheung et |
| | on PLA - Silk | PLA | property tests: | properties | applications | al 2012 |
| | fiber composites | | Tensile property | Thermal properties, | | |
| | for | | FTIR, | Biodegradability | | |
| | prosthetic | | scanning laser | enhanced | | |
| | applications | | extensometers | | | |
| | | | Microscopic | | | |
| | | | analysis: silk | | | |
| | | | dispersion and | | | |
| | | | orientation of silk | | | |
| | | | fiber | | | |
| 7 | Citric acid based | POC(Poly(1,8- | Biomineralization, | Bioactive, | For interference | Honglin Qiu et |
| | hydroxyapatite | octanediol-co- | in-vivo | Biocompatible and | screws | al 2006 |
| | composite for | citrate) HA with | biocompatibility | biodegradable | | |
| | orthopedic | percentage of | and in-vitro | biocomposites | | |
| | implants | 40, 50, 60 and 65% | degradation | | | |
| 8 | General | Types of soft | Biodegradable and | Biodegradable | For soft tissue | Ronald lakstos |
| | principles of | flxation devices | biocompatibility | fixation | fixation devices | et al 2009 |
| | internal fixation | like screws, pins, | studies | biocompatibility | like ACL, | |
| | | staples and sutures | | | meniscal repair | |
| 9 | Poly-L-lactic | PLLA/HA | Biocompatibility, | MRI to assess | For ACL fixation | L. macarini et al |
| | acid- | | osteoconductivity | degradatiion | | 2008 |
| | hydroxyapatite | | and | | | |
| | bioreabsorbable | | biodegradation | | | |
| | interference | | | | | |
| | screws for tibial | | | | | |
| | graft fixation in | | | | | |
| | anterior cruciate | | | | | |
| | ligament | | | | | |
| | reconstruction | | | | | |
| | surgery | | | | | |
| 10 | Long-term | PLLA/β-TCP | Osteoconductive, | Radiography, CT | For ACL fixation | F. Alan barber et |
| | Absorption of β- | 75% and 25% | Biomechanical | scan | | al 2007 |
| | tri-calcium | | properties | | | |
| | phosphate poly- | | | | | |
| | L-lactic acid | | | | | |
| | interference | | | | | |
| | screws | | | | | |
| 11 | Bioadsorbable | 15% TCP and | Biocompetibility | — | For tendon to | Shane J. Nho et |
| | anchors in | 85% PLA | and mechanical | | bone fixation | al 2008 |
| | glenobumeral | | strength | | | |
| | shoulder surgery | | | | | |
| 12 | Biomechanical | Surgical | Tensile testing | — | Cancellous | Mesahiro |
| | composition of | techniques used | pull out strength | | screw resulted in | Kurosaka et al |
| | different surgical | staple fixation, | | | higher stiffness | 1987 |
| | techniques of | tying sutures, over | | | used in petellar | |
| | graft fixation is | buttons and screw | | | tendon graft. | |
| | anterior cruciate | fixation | | | | |
| | ligament | | | | | |
| | reconstruction | | | | | |
| 13. | Bioabsorbable | PLLA | Cyclic loading | Mechanical | Tibial insertion | Vladimir |
| | sutures versus | | pull out and | strength | of ACL with | senekovic et al |
| | screw fixation of | | tensile strength | | arthroscopic | 2014 |
| | displaced tibial | | | | reduction | |
| | eminence | | | | | |
| | fracture: a | | | | | |
| | biomechanical | | | | | |
| | study | | | | | |
| 14. | Mechanical | PLLA | Biomechanical | Mechanical | Absorbable | Alberto G. |
| | Strength of | | tests: Cyclic | strength | sutures for | Schneeberger et |
| | Arthroscopic | | loading, pull out | | rotator cuff | al 2002 |
| | Rotator Cuff | | and tensile | | | |
| | Repair | | strength | | | |
| | Techniques | | | | | |

With the thorough patent and non patent literature survey, it has been observed that the marketed products prepared from PLLA, PLGA, and PDLLA suffer from following drawbacks:
1. Release of acidic degradation byproducts responsible for inflammatory (immune) reactions
2. Poor bioactivity
3. Mechanical properties mismatch, viz. Young's modulus not matching with plateau of hysteric behavior of soft tissue viz. ligament, tendons, etc., resulting in tissue loosening limiting its applications in ligament and tendon tears repair
4. High melting point which makes processing difficult

Further, none of the cited prior arts matches the ideal properties for implant for fixation which provides substantial efficacy for bone and soft-tissue fixation and also gives full resistance against all bacterial infections at the site of implant.

Hence, there is a long-felt need for preparing of improved fixation devices in world orthopedic market driven by the increasing demographics aging population across the world), as there is direct correlation between fractures and elderly population according to National Health Statistics Report published in 2010 by CDC (Centre for disease control and prevention). Metals, ceramics, polymers, composites, etc. have been explored as materials for orthopedic devices, but, very few have matched ideal properties of implant for fixations, thus there is still need and scope of improvement in the current materials.

The present invention meets the above-mentioned long-felt need.

### OBJECTS OF THE INVENTION

The principal object of the present invention is to provide a novel bioresorbable polymer-composite, which is used to make orthopedic devices to cater fixation of soft-tissue injuries, small bone fractures and fractures in pediatrics.

Another object of the present invention is to provide a novel bioresorbable polymer-composite for bone-soft tissue fixation which allows bone tissue proliferation and supports vascularization.

Yet another object of the present invention is to provide a novel bioresorbable polymer-composite for bone-soft tissue fixation which provides better biocompatibility and osteo conduction.

Further object of the present invention is to provide a novel bioresorbable polymer-composite for bone-soft tissue fixation which is biocompatible and resorbable.

Another object of the present invention is to provide a novel bioresorbable polymer-composite for bone-soft tissue fixation which is economic thus reaching to mass population.

### BRIEF DESCRIPTION OF THE ACCOMPANYING DRAWINGS

FIG. 1 shows a schematic representation of test biomaterial compositions prepared by micro-compounding and injection molding.

FIG. 2 shows the comparative analysis of tensile strength data of test samples.

FIG. 3 shows tensile modulus data of PCL and silk-PCL (5, 10, 20, 30, 40% filler) composites.

FIG. 4 shows a schematic of methodology for % hemolysis ratio assay.

FIG. 5 shows % Hemolysis ratio sample after incubation of test bioniaterial composites with human blood for 4 hours. A—Saline (Negative), B—5% silk-PCL, C—10% silk-PCL, D—20% silk-PCL, E—30% silk-PCL, F—40% silk-PCL, G—0.1% Triton-X (positive).

FIG. 6 shows Microscopic images of A) Negative control and 40% silk-PCL B) Positive (Triton-X treated).

FIG. 7 shows % Hemolysis ratio of test biomaterial compositions (silk-PCL composites) compared to negative and positive control.

FIG. 8 shows a schematic of methodologies for APTT and PT assays; (A) preparation of platelet poor plasma (PPP), (B) and (C) Sequential steps in APTT and PT assay using PPP.

FIG. 9 shows a prothrombin time of test biomaterial compositions (silk-PCL composites) compared to negative control (physiological saline).

FIG. 10 shows a schematic of methodologies for platelet count (PC) assay; (A) Preparation of platelet rich plasma (PPP), (B) Sequential steps in PC assay using PRP.

FIG. 11 shows an effect of different test biomaterial compositions (silk-PCL composites) on platelet count compared to negative control (physiological saline) and positive control (0.1% Triton-X) after incubation with human blood.

FIG. 12 shows hemocompatibility data: (A) % hemolysis and B platelet count values for test samples (MgO-silk-PCL composites).

FIG. 13 shows hemocompatibility data: (A) aPTT and (B) PT values for test samples; M1—5% silk-10% MgO-PCL, M2—5% silk-20% MgO-PCL, M3—10% silk-10% MgO-PCL, M4—10% silk-20% MgO-PCL, M5—20% silk-10% MgO-PCL, M6—20% silk-20% MgO-PCL, M7—Negative Control=Saline, M8—Positive control=heparin for aPTT.

FIG. 14 shows as-molded dog bone-shaped tensile testing specimen of silk-PCL composites (ASTM D-638 type V).

FIG. 15 shows As-molded dog bone-shaped tensile testing specimens of MgO-silk-PCL composites (ASTM D-638 type V).

### DETAILED DESCRIPTION OF THE INVENTION

The present invention discloses a novel bioresorbable, biocompatible polymer composite for bone soft tissue fixation which can be used to prepare different orthopedic devices which eventually cater fixation of soft tissue injuries, small bone fractures, fractures in pediatrics etc.

The polymer composite is preferably composed of blend of bioresorbable polymer such as poly-ε-caprolactone (PCL), natural fiber silk fibroin and an osteo conductive component like Magnesium oxide (MgO) in nanoparticle form.

Among them, natural fiber silk fibroin and MgO have been added as filler. However, the mechanical, thermal and degradation properties can be customized by the use of natural fiber silk fibroin which is extracted from Bombyx mori.

The ingredients used in this composition are FDA-approved.

The detailed composition along with weight percentages has been given below:

i) Magnesium Oxide (MgO) or other ceramic fillers→5 to 30%

ii) Silk fibroin or other natural fibers→5 to 30%

iii) a polymer matrix such as polycaprolactone and other bioresorbable polymers→40 to 90%

FIG. 1 illustrates this composition with the block diagram.

Hence, protection for degummed silk composition (5% to 30%) with or without MgO nanoparticles composition (5% to 30%) (or other ceramic fillers like HA, β-TCP, SiO₂, CaO, CaCO₃, etc. in Polycaprolactone-quantity sufficient to 100% (or other bioresorbable polymer viz. PLLA, PLGA, etc.) is sought.

The tunability in mechanical properties, degradation rate and bioactivity/biomineralization is desired for different bone-soft tissue fixation applications which could be achieved by varying filler concentrations (MgO nanoparticles and silk fiber) viz. for low load bearing applications like soft tissue fixations lower mechanical strength is desired as compared to high load bearing applications viz. pediatric or small bone fracture fixations, etc. This could be achieved by varying filler concentration.

In this composition, PCL has been used a main polymer matrix which has some advantages over conventionally used PLLA, PLGA.

i) Mechanical property (Young's modulus) of human tissues viz. Cancellous bone, ligament, tendon, etc. ranges from 0.02-2.31 GPa, most of polymers viz. PLLA, PLGA, etc. ranges from 2-3 GPa which is at the upper limit of required range while that PLC is 0.2-0.5 GPa which can be tuned to match required mechanical properties by filler reinforcement with ceramic particles, natural fiber, etc. or ratio of polymers in polymer blends (PCL:PLLA/PLGA) can be varied to achieve desired mechanical profiles.

ii) lower melting point makes its processing easier

iii) It is nontoxic, biocompatible polymer

iv) It produces non-inflammatory degradation products like water and carbon dioxide

v) Environmental-friendly

vi) Good thermoplastic and mold ability

vii) Good compatibility with wide range of polymers

The PCL owing to high degree of crystallinity lowers the degradation that limits its application, however, its degradation rate can be tailored by addition of hydrophilic fillers which is in-turn responsible for polymer composite undergoing degradation by both bulk and surface erosion (unlike, only surface erosion in case of neat polymer), hence, enhanced degradation rate. Its mechanical properties, degradations kinetics, bioactivity, etc. are tailorable based on filler concentration.

Magnesium oxide nanoparticles have been incorporated in FDA-approved biocompatible polymers (like PLLA) to formulate composite biomaterials imparting improvement in mechanical and biological properties of neat polymer for various biomedical applications.

Some of the representative examples MgO nanoparticles as ceramic filler are given below:

1) surface modified magnesia (g-MgO) nanoparticles (1, 2, 3, 4, 5% w/w) loaded PLLA composites with improved mechanical and biological properties in-vitro,

2) MgO-Polystyrene composite (5, 10, 15% MgO w/w) to improve mechanical (tensile strength and modulus) properties of composites.

3) 10% and 20% MgO w/w in PLLA with and without 10% HA w/w to improve in-vitro biological performance (osteoblast adhesion and proliferation, biodegradation) of composite.

Further, natural silk fibroin which is extracted from Bombyx mori also improves mechanical properties of PCL.

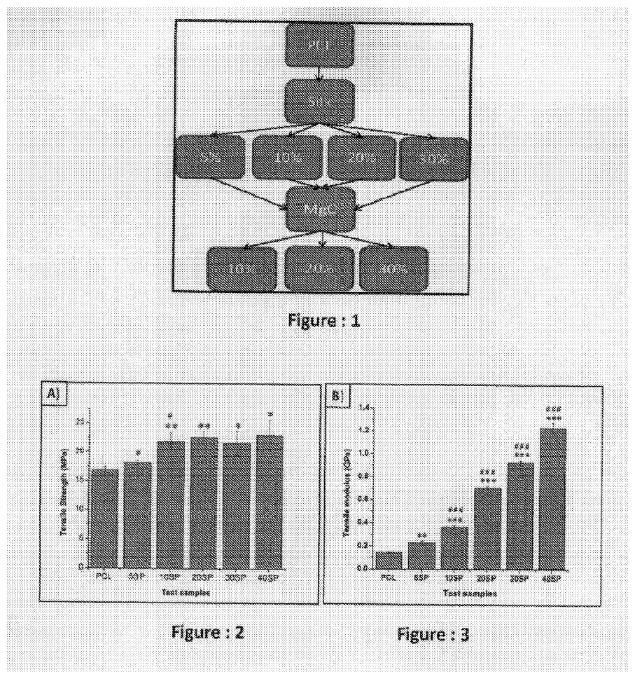

According to FIG. 2, the polymer composite has also characterized in substantially enhanced tensile properties (strength and modulus) and hence, some elaborate testing has been done so far.

To investigate mechanical properties of different fiber-polymer composites, dog-bone shaped tensile testing specimens were prepared according to ASTM standards (D638 TypeV). Mechanical properties (Tensile strength, tensile modulus) were extracted from stress-strain data and compared to understand the effect of increasing filler addition on mechanical behavior of silk-PCL composites.

FIG. 2 illustrates comparative analysis of tensile strength data of test samples (N=3).

From FIGS. 2 and 3 there are provided (A) Tensile strength data of PCL and silk-PCL (5, 10, 20, 30, and 40% filler) composites, P<0.05*, P<0.01**, P<0.001***, PCL Vs silk-PCL composites and P<0.05#, 5% Silk-PCL Vs 10% Silk-PCL; (B) Tensile modulus data of PCL and silk-PCL composites, P<0.05*, P<0.01**, P<0.001***, PCL Vs silk-PCL composites and P<0.05 #, P<0.01 # #, P<0.001 # # #, silk-PCL composites Vs 5% silk-PCL composite (SP=Silk-PCL, prefix digit=filler concentration).

Hence, from FIGS. 2 and 3, it can be concluded that there was significant improvement in tensile properties by addition of filler fibers with respect to neat polymer. Tensile strength increased approximately 2 times whereas modulus increased 10 folds with 40% filler, which was highest among all samples tested. Increase in tensile properties may be attributed to removal of hydrophilicity on native silk surface due to seric in by degumming, thus rendering silk fiber hydrophobic (fibroin), which helps in better interfacial bonding between filler and hydrophobic polymer matrix, thus, improving mechanical properties.

MgO nano-particles are explored as potential bioactive fillers to impart bioactivity, in addition to improving mechanical properties of PCL and taking advantage of its unique antibacterial property to combat against microbes responsible for implant related infections.

Although MgO has been considered as preferred bio-ceramic material in the present invention, other ceramic materials like HA, silicon dioxide, calcium carbonate, calcium oxide, calcium trisilicate, Magnesium calcium trisilicate, calcium containing compounds such as mono, di, octa, tri calcium phosphate and mixture thereof may also be used. Composition of the present invention may also contain a bioactive glass comprising metal oxides such as calcium oxide, silicon dioxide, sodium oxide, etc. and mixture thereof.

Thus, present biocomposite is blend of bioactive nanofiller viz. MgO, HA, etc. and silk fibroin in bioresorbable FDA-approved polymer matrix viz. PCL, PLLA, etc. or mixture thereof.

Biocomposites have been widely used in orthopedic application due to their biocompatibility, osteo conductivity and mechanical stability of the implants. However, implantation of such biocomposites leads to damage of bone matrix due to increase in bone resorption as it may imbalance the bone remodeling, followed by an inflammatory response which in turn induces implant loosening as a biological consequence of particulate debris.

To overcome this disadvantages bisphosphonates (BPs) analogues have been used as coating onto implant or incorporation in polymer matrix would inhibit osteolysis in the vicinity of implants by reacting directly with osteoclasts according to the present invention, Antibiotics may also be incorporated to treat osteomyelitis and inflammation at the site of implants.

MgO filler may also impart antibacterial and anti-bone-resorption activity to biocomposite to eliminate need of antibiotic and bis-phosphonate coating to bone implants.

The preparation of individual ingredients is as follows:

### Materials and Methods

### Materials Used in Fabrication

1. Degummed silk: i) Silk cocoons Bombyx mori were procured from silkworm rearing farmer associated with Research Extension Centre, Central Silk Board C/o: District Sericulture Development Office, Yashatara Bunglow, Near Janade Saw Mill, Dwarka Circle, Nasik (Maharashtra)-422001, (more information can be found at Regional Office, Central Silk Board, No. 16, Second Floor, Mittal Chambers, Nariman Point, Mumbai-400021, Maharashtra), ii) sodium carbonate purchased from sigma Aldrich and iii) ultrapure water.

2. Poly-ε-caprolactone (molecular weight 80,000) was purchased from Sigma Aldrich (Germany).

3. Magnesium oxide nanoparticles were synthesized using i) Magnesium chloride salt (SD chemicals, Mumbai), ii) NaOH (SD chemicals, Mumbai).

### Methods and Procedure Used in Preparation Degumming of Silk Cocoons

Following protocol was followed given by Kaplan et at to remove sericin from silk fibroin:

i. Degummed silk fibers were prepared by processing Bombyx mori silk cocoons. 5-litres beaker was filled with 2 liters of ultrapure water and covered with aluminum foil followed by heating till boiling.

ii. Measured quantity of 0.02 M sodium carbonate was added to the boiling water and stirred thoroughly to dissolve completely.

iii. Cocoons were added to boiling sodium carbonate solution and stirred for 30 mins.

iv. After boiling, silk fibroin was removed with spatula and cooled by rinsing in ultrapure cold water, excess water squeezed out of the silk.

v. Silk fibroin was then rinsed in 1 liter of water for 20 min with stirring on a stir plate.

vi. Steps 4 and 5 were repeated twice for a total of three rinses.

vii. After the third wash, silk fibers were removed, squeezed well and then spread on a clean piece of aluminum foil.

viii. Silk fibroin eras allowed to dry in a fume hood overnight.

ix. Dried degummed silk fibroin was chopped to length of around 5-10 mm to use as filler.

### Preparation of Magnesium Oxide Nanoparticles:

Magnesium oxide nanoparticles synthesis was carried out using simple hydroxide precipitation method.

i. Magnesium chloride salt (SD chemicals, Mumbai) solution (1 mol/L) was added to alkaline solution of NaOH (SD chemicals, Mumbai) (2 mol/L)

ii. It was stirred vigorously for 3 hr on water bath/hot plate, reaction mixture temperature maintained at 80° C.

iii. On precipitation white colored Magnesium hydroxide formed in mother liquor was allowed age at room temperature for 1 day.

iv. After aging, suspension was centrifuged at 10,000 rpm for 10 mins at 15° C.,

v. Supernatant was decanted and fresh MiliQ water added to give washing for 3 times followed by ethanol washings.

vi. Precipitate was then dried in oven for 4 hrs at 60° C.

vii. Dried sample was then subjected to hydrothermal treatment i.e. heated to 250° C. for 1 hr, 370° C. for 2 hrs and 450° C. for 3 hrs, to remove water molecule and obtain MgO nanoparticles from Mg(OH)₂

Fabrication of Composite with Micro-Compounding and Injection Molding PCL-Silk Composite

Micro-compounding (twin-screw extrusion) was selected as method of composite fabrication, because it: (i) ascertains uniform distribution and dispersion of the filler during mixing and, hence, more uniform nucleation sites for bioactivity, and (ii) provides an environment-friendly manufacturing method eliminating solvents, thus minimizing inflammatory in-vivo responses.

All the degummed silk fibers were chopped into 5-10 mm in length in order to avoid coiling with the micro-compounder screws and pre-dried for 24 hours at 50° C. to remove traces of moisture. Silk fiber/PCL composite samples were made using the Xplore DSM 5 cm³ twin-screw micro-extruder.

The silk fibers in different filler concentrations 10%, 20%, 30%, and 40% were used for melt-mixing with PCL. A uniform temperature of 160° C. was maintained at all mixing zones inside the micro-compounding machine. The operating conditions of the micro-compounder were set as screw speed, mixing temperature and mixing time at 150 rpm, 160° C. and 15 mins, respectively. Pre-weighed quantities of silk fibers and PCL were fed into the twin-screw extruder. At the end of mixing period, the extrudate was collected in Piston Cylinder that fits into injection molding machine (Xplore DSM 5 cm³). Injection molding was carried out with processing parameters viz. cylinder temperature, mold temperature and pressure set at 160° C., 30° C. and 3 bars, respectively. Tensile testing specimens were prepared in a dog bone-shape according to ASTM D638 type V (FIG. 1).

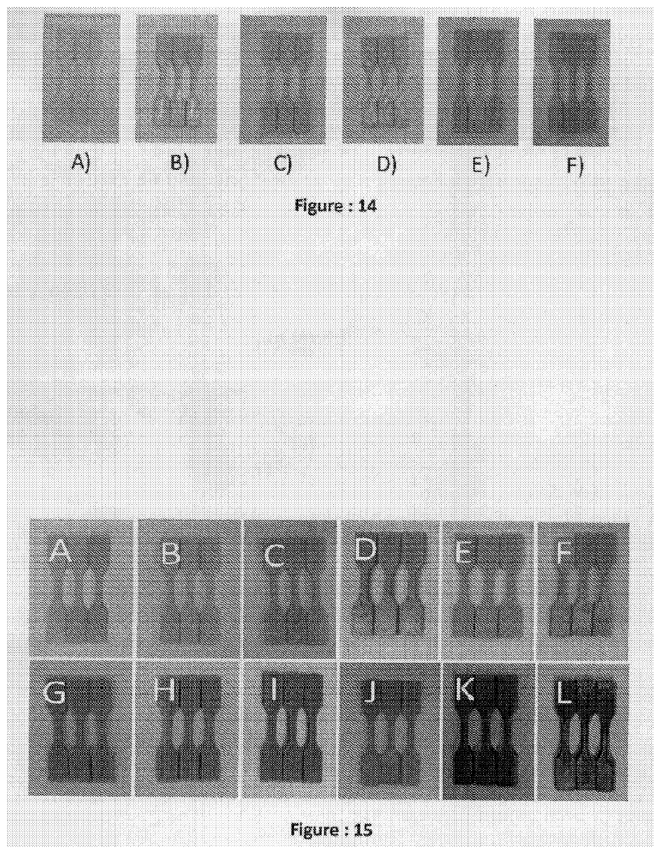

FIG. 14 shows as-molded dog bone-shaped tensile testing specimen (ASTM D-638 type V) A) PCL, B) 10% Silk-PCL, C) 20% Silk-PCL, D) 30% Silk-PCL and E) 40% Silk-PCL

### MgO-Silk-PCL Composite

Before mixing, silk fibers were chopped into 5 mm fibers, MgO nanoparticles powder was pre-dried to remove moisture traces before melt-mixing.

MgO filler in concentration of 10%, 20% and 30% were mixed with silk fiber concentrations 5%, 10%, 20%, and 30% (FIG. 1) in PCL polymer matrix quantity sufficient to make 100% w/w. Thus, total of 12 sets of MgO-silk-PCL composites were prepared and one set of PCL alone for comparison analyses (FIG. 15) using micro-compounder and injection molding machine to obtain tensile specimens. These specimens were then subjected to various analyses to assess their potential for orthopedic biomaterial applications.

FIG. 14 illustrates molded As-molded dog bone-shaped tensile testing specimen of silk-PCL composites (ASTM D-638 type V); (A) PCL, (B) 5% Silk-PCL, (C) 10% Silk-PCL, (D) 20% Silk-PCL, (E) 30% Silk-PCL, (F) 40% Silk-PCL.

FIG. 15 illustrates different compositions for molded dog bone-shaped tensile testing specimen of MgO-silk-PCL composites (ASTM D-638 type V).

1) 5% silk-PCL:

A) 5% silk-10% MgO-PCL,

B) 5% silk-20% MgO-PCL,

C) 5% silk 30% Mgo-PCL,

2) 10% silk-PCL:

D) 10% silk-10% MgO-PCL,

E) 10% silk-20% MgO-PCL,

F) 10% silk 30% MgO-PCL,

3) 20% silk-PCL:

G) 20% silk-10% MgO-PCL,

H) 20% silk-20% MgO-PCL,

I) 20% silk-30% MgO-PCL,

4) 30% silk-PCL:

J) 30% silk-10% MgO-PCL,

K) 30% silk-20% MgO-PCL,

L) 30% silk 30% MgO-PCL

### Some Clinical Test Results to Show the Enhanced Efficacy for the Bioresorbable Polymer Composite Used in the Present Invention.

The various test results for the novel bioresorbable composition have been given below:

Though, preclinical tests on rabbit models are in-progress to prove biosafety of as-developed orthopedic biomaterial, inventers could successfully perform hemocompatibility tests on said biomaterial compositions with human blood according to with prior permission from institute ethics committee and institute biosafety committee. Three parameters were assessed to check if biocomposite is harmless to human blood cells and doesn't affect its coagulation process adversely.

A) % Hemolysis ratio: To evaluate amount of erythrocyte lysis when test biomaterial is incubated in presence of human blood.

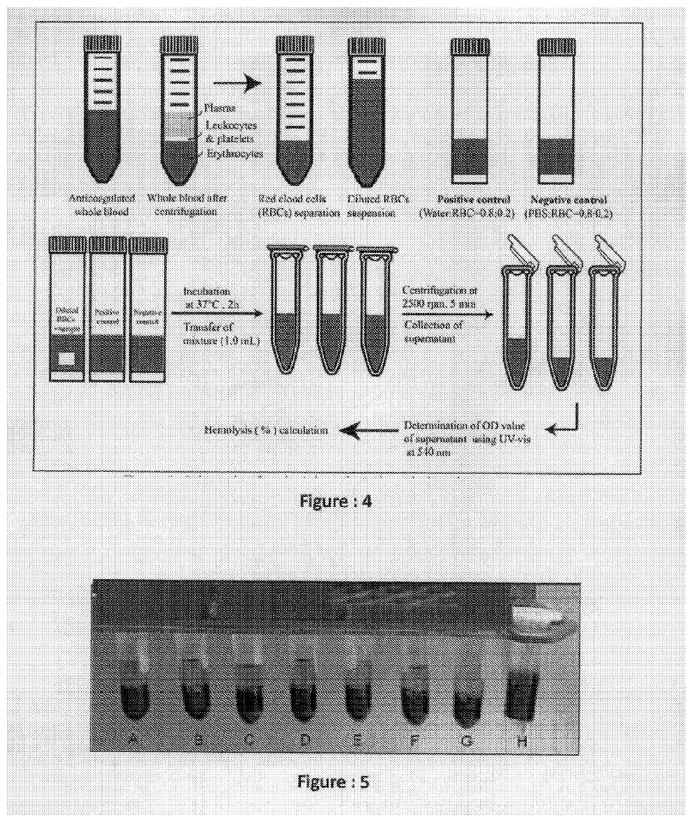

FIG. 4 illustrates schematic of methodology for % hemolysis ratio assay

FIG. 5 illustrates % Hemolysis ratio sample after incubation of test biomaterial composites with human blood for 4 hours.

A=Physiological saline (Negative control), B=PCL, C=5% silk-PCL, D=10% silk-PCL, E=20% silk-PCL, F=30% silk-PCL, G=40% silk-PCL and H=0.1% Triton-X (positive control). Red color of supernatant indicates hemolysis (positive control); Representative optical microscopic images of erythrocytes in blood incubated with (B) Test sample (40% Silk-PCL), (C) Negative control and (ID) Positive control, scalebar=50 μm.

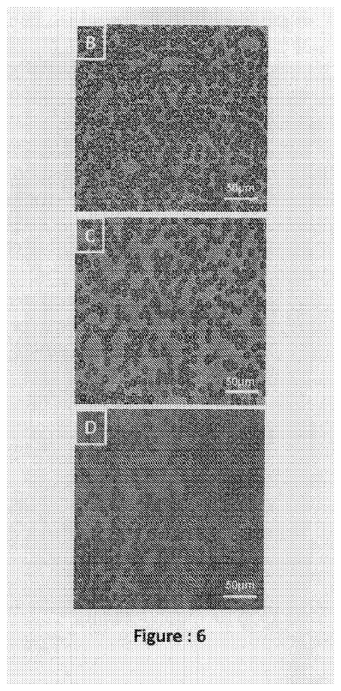

The microscopic images of A) Negative control and 40% silk-PCL B) Positive (Triton-X treated) has been illustrated by FIG. 6.

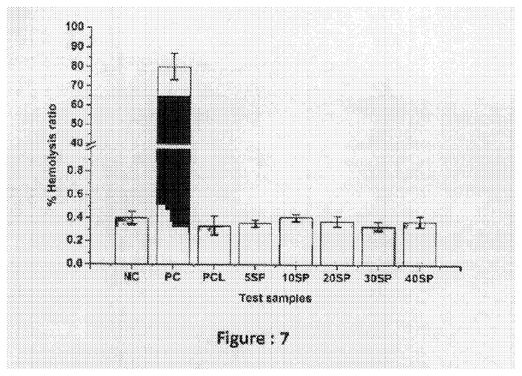

FIG. 7 illustrates % Hemolysis ratio of test biomaterial compositions (silk-PCL composites) compared to negative and positive control.

B) Activated Partial Thromboplastin Time (APTT) and Prothrombin Time (PT):

Blood plasma APTT and PT tests are commonly used to evaluate the effect of test biomaterial on blood coagulation properties.

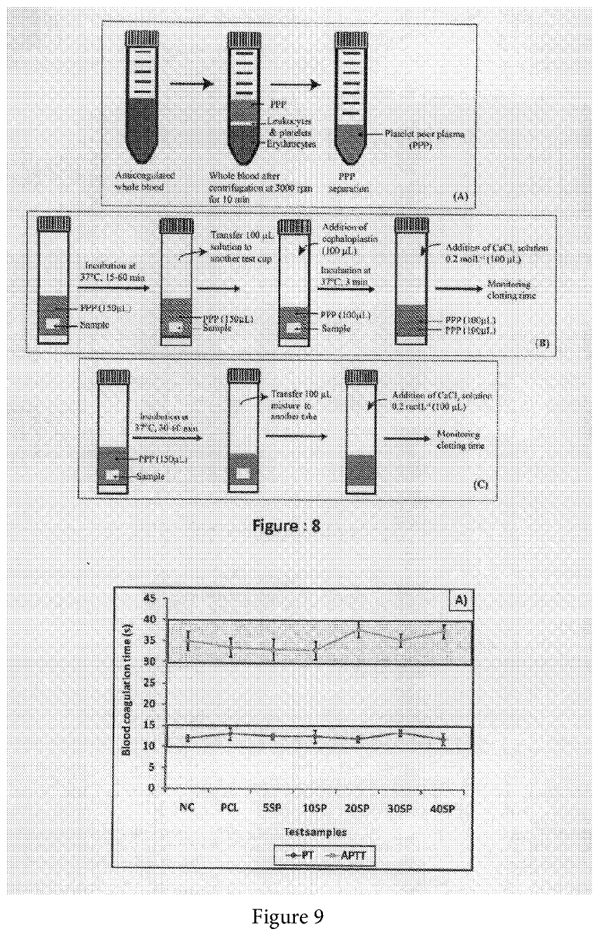

FIG. 8 illustrates a schematic of methodologies for APTT and PT assays; (A) preparation of platelet poor plasma (PPP), (B) and (C) Sequential steps in APTT and PT assay using PPP.

A prothrombin time of test biomaterial compositions (silk-PCL composites) compared to negative control (physiological saline) has been shown in FIG. 9.

From FIG. 9, it shows that (A) Dynamic blood clotting time (s): Prothrombin time (PT) and activated partial thromboplastin time (APTT) of blood samples treated with test biomaterial samples (PCL and SP=Silk-PCL composites and prefix digit=filler concentration) compared to NC=negative control (physiological saline);

C) Platelet count (PC): To study the effect of biomaterial on platelet count. If biomaterial surface promotes platelet activation, it may lead to platelet adhesion/aggregation (finally thrombosis), hence, reduction in platelet count.

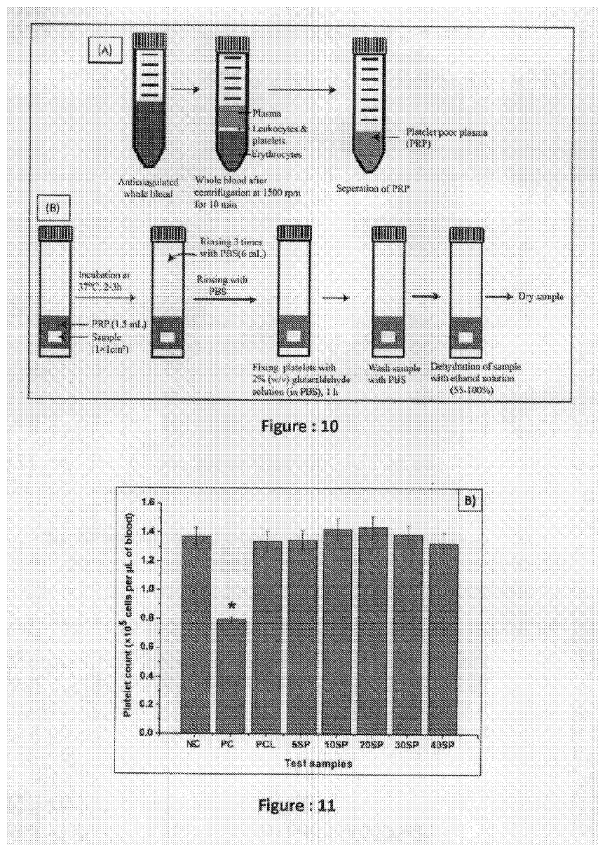

FIG. 10 illustrates as chematic of methodologies for platelet count (PC) assay; (A) Preparation of platelet rich plasma (PPP), (B) Sequential steps in PC assay using PRP.

According to FIG. 11, an effect of different test biomaterial compositions (silk-PCL composites) on platelet count compared to negative control (physiological saline) and positive control (0.1% Triton-X) after incubation with human blood has been illustrated.

### Hemocompatibility Studies Data of MgO-Silk-PCL Composite Biomaterial:

Hemocompatibility of test samples was assessed on human blood with test parameters such as % hemolysis ratio, platelet count, activated partial Thromboplastin time and Prothrombin time.

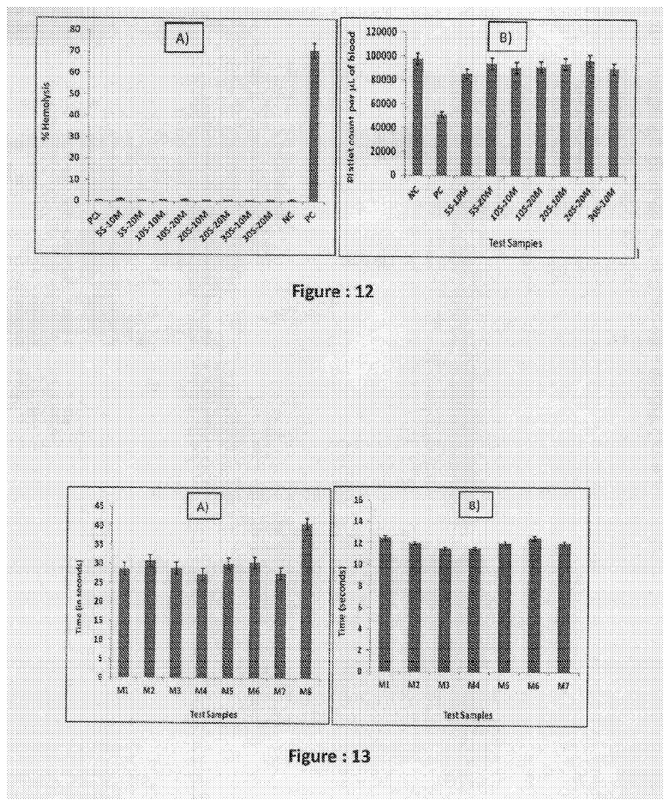

FIG. 12 illustrates Hemocompatibility data: (A) % hemolysis and (B) platelet count values for test samples MgO-silk-PCL composites.

From FIG. 13, Hemocompatibility data: (A) aPTT and (B) PT values for test samples; M1—5% silk-10% MgO-PCL, M2—5% silk-20% MgO-PCL, M3—10% silk-10% MgO-PCL, M4—10% silk-20% MgO-PCL, M5—20% silk-10% MgO-PCL, M6—20% silk-20% MgO-PCL, M7—Negative Control=Saline, M8—Positive control=heparin for aPTT.

### Results and Discussion:

All the test compositions showed no harmful effect on blood coagulation properties as Prothrombin time (9-15 seconds) and activated partial Thromboplastin time (25-35 seconds) are both within normal range, also, it doesn't affect blood cells adversely as % hemolysis ratio for all test composites is below 0.5% (<1%: Non-hemolytic, 1-3%: mild, 3-5: moderate and >5% severely hemolytic) and platelet count is also within normal range i.e. 1.5-3.5×105 cell/μL, of human blood (FIGS. 10 and 11).

Thus, all observations by studying various parameters for hemocompatibility using human blood indicate that biomaterial compositions under investigation are hemo-compatible i.e. do not interfere with normal blood cell viability, count, coagulation process, etc. and suitable for biomedical use involving human blood contact.

### The Non-Limiting Advantages of the Present Invention are as Follows:

(1) Tunable mechanical, biological properties (as per filler loading) for wider clinical applications;

a) Matchable strength to bone or soft-tissue owing to silk fiber and MgO reinforcement: no stress-shielding

b) Higher biomineralization and biocompatibility

c) Tailorable biodegradation to match bone or soft tissue healing rate

(2) Localized infection resistant due to antibacterial properties of MgO nanoparticles

(3) Anti-resorption ability Mg²⁺ ions helping in proper bone remodeling

(4) No local inflammatory reactions (Like PLLA implants) due to neutralization effect of alkaline Mg²⁺ ions on acidic degradation byproducts of PCL

(5) Economic biomaterial composite owing to use of inexpensive/easily available/synthesizable raw material and well-established manufacturing process

The present composition can be used in wide range of process that can encompass any type of tissue modification (hard tissue like bone and/or soft tissue like tendon, ligament, etc.), including tissue repair, reconstruction, remodeling, also includes in the processes that affect the orifice such as mouth and nose (e.g. the composition described herein can be used in dental procedures).

The present invention is not limited to the human patients; it can be very well employed in developing bioresorbable orthopedic devices for veterinary applications addressing different bone anomalies in animals viz. pets (e.g., dogs and cats), farm animals (such as goats, sheep, cow, pigs, horses), laboratory animals (rodents like rats and mice and non-rodents such as rabbits) and wild animals.

1.-8. (canceled)

9. A novel bioresorbable polymer composite for bone soft tissue fixation comprising:
i) 5-30% silk fibroin;
ii) 40-90% bioresorbable polymer matrix; and
iii) 5-30% magnesium oxide or other ceramic fillers.

10. The novel bioresorbable polymer composite as claimed in claim 9, wherein the silk fibroin is extracted from Bombyx mori and degummed using a Na₂CO₃ hot bath method.

11. The novel bioresorbable polymer composite as claimed in claim 9 comprising at least one bioresorbable polymer selected from the group consisting of Polycaprolcatone (PCL), poly (L-lactide) (PLLA), poly (D,L-lactide) (PLDLA), poly (lactide-co-glycolide) (PLGA), poly (glycolide-co-trimethylene carbonate) (PGA-TMC), Polydioxanone (PDO).

12. The novel bioresorbable polymer composite as claimed in claim 9, wherein the said filler is selected from Magnesium Oxide (MgO), Hydroxyapatite (HA), β-Tricalcium phosphate (β-TCP), silicon dioxide (SiO₂), Calcium oxide (CaO), Calcium Carbonate (CaCO₃), calcium trisilicate, Magnesium calcium trisilicate, calcium monophosphate, calcium diphosphate, calcium triphosphate, and calcium octaphosphate.

13. The polymer composite as claimed in claim 9, further comprising a bioactive glass comprising a metal oxide.

14. A method of preparing the polymer composite as claimed in claim 9, comprising the steps of:
chopping silk fibers into small pieces;
mixing the chopped silk fibers, MgO nanoparticle powder, and pre-dried PCL polymer pellets;
mixing of different concentration of MgO filler with silk fibers in different concentrations to obtain the bioresorbable polymer composite.

15. The method as claimed in claim 14, wherein said concentration of silk fibers in the polymer composite is from 5% to 30%.

16. The method as claimed in claim 14, wherein mixing of silk fibers and MgO filler with PCL takes place at a speed of 100-200 rpm, duration of 10-20 min and temperature of 140-180° C.
